(19) United States
(12) Patent Application Publication
(10) Pub. No.: US 2015/0045429 A1
(43) Pub. Date: Feb. 12, 2015

(54) HYDRATING COMPOSITION, USE OF A COMPOSITION AND COSMETIC FORMULATION CONTAINING A HYDRATING COMPOSITION

(71) Applicant: OXITENO S.A. INDÜSTRIA E COMÉRCIO, São Paulo, SP (BR)

(72) Inventors: Lêda Fernanda de Jesus Oliveira, Sao Paulo (BR); André Luis Conde Da Silva, Sao Paulo (BR)

(73) Assignee: OXITENO S.A. INDÜSTRIA E COMÉRCIO, São Paulo, SP (BR)

(21) Appl. No.: 14/377,703

(22) PCT Filed: Feb. 6, 2013
(86) PCT No.: PCT/BR2013/000039

§ 371 Date: Aug. 8, 2014

(30) Foreign Application Priority Data
Feb. 10, 2012 (BR) 10 2012 003136 1

(51) Int. Cl. A61K 8/86; A61Q 19/00

(52) U.S. Cl. CPC A61K 8/86; A61Q 19/00; A61K 2800/5922
USPC 514/547

(57) ABSTRACT

The present invention describes a new moisturizing composition comprising a mixture of esters of one or more alkoxylated polyols and finds use in the formulation of cosmetics for the skin. The composition of the present invention can be in the solid form or in the liquid form, in which case is also comprises one or more surfactants, emollients and, optionally, water. The moisturizing composition described herein is compatible with a large variety of emollients and additional ingredients used in cosmetic formulations, and has shown to be able to maintain the skin hydrated for 24 hours.

### FIELD OF THE INVENTION

The present invention discloses a moisturizing composition for cosmetic products. Specifically, this invention describes a composition comprising a mixture of esters of one or more alkoxylated polyols having the ability of moisturizing the skin as well as a cosmetic formulation comprising the composition.

### BACKGROUND OF THE INVENTION

The skin is formed by three layers, the innermost hypodermis, the intermediate dermis and the outer-most layer epidermis. For the skin to remain healthy, it is important for the top layer of the epidermis, the stratum corneum, to remain moisturized. Skin hydration means the relative increase in the water content, as determined by the corneometry test (U. Heinrich et al., “Multicentre comparison of skin hydration in terms of physical-, physiological- and product-dependent parameters by the capacitance method—Corneometer CM 825”, International Journal of Cosmetic Science, 25, 45-51, 2003). In addition to conferring flexibility to the skin and to avoid desiccation and the appearance of cracks, hydration is also important for preventing chronic diseases.

The most common way to moisturize the skin is through the use of moisturizing cosmetic formulations. These formulations are usually in the form of body butters, creams and lotions or skin milks, which basically differ from each other by their consistencies. In general, these formulations comprise emulsions that can be oil-in-water (O/W), where water is the outer phase of the emulsion, or water-in-oil (W/O), with oil being the outer phase. Some formulations are also presented in the form of micro- or nanoemulsions, having greater stability.

Cosmetic formulations may be formed by a large variety of components, including one or more (i) emollients, such as oils or silicones, responsible by the replacement of skin lipids; (ii) emulsifiers, commonly one or more surfactants, having as their primary function to impart stability to the formulation; (iii) consistency agents, which can be gums or polymers used to adjust viscosity; and (iv) further components, such as vitamins, antioxidants and moisturizers. Glycerin is the most widely used compound as a moisturizer in cosmetics (S. Verdier-Sévrain, F. Bonté, “Skin hydration: a review on its molecular mechanisms”, Journal of Cosmetic Dermatology, 6, 75-82, 2007), but compounds of different chemical natures may present such an action, including certain polymers. Cosmetic formulations with moisturizer action having polymers belonging to distinct chemical classes, such as polysaccharides, amino acids or synthetic polymers, have been devised (US20040191205; US20070202070; L. Chrit et al., “In vitro and in vivo confocal raman study of human skin hydration: assessment of a new moisturizing agent, pMPC”, Biopolymers, 85, 359-369, 2006).

From among the different types of polymers used in cosmetic formulations, alkoxylated polyol derivatives stand out, which have low toxicity, do not irritate the skin and can impart other desirable characteristics to a cosmetic composition, including suitable consistency and pleasant sensory characteristics. However, apparently the use of such a polymer as a moisturizer in cosmetics has been little explored so far. One example was found to be the North American invention U.S. Pat. No. 4,687,843 (“Esterified propoxylated glucose compositions”, 1987), which describes the use of propoxylated methyl glucoside esters in cosmetic products in order to moisturize the skin. In U.S. Pat. No. 4,687,843, these compounds were obtained by combining from 5 to 50 moles of propylene oxide per each mole of methyl glucoside and further reacting the propoxylated derivative with saturated or unsaturated fatty acids, the formation of diesters being preferred. Nevertheless, taking into account the relative low propoxylation degree, one cannot expect the esters of the invention disclosed in the North American U.S. Pat. No. 4,687,843 to exhibit superior moisturization or even thickening properties.

During recent tests to check the thickening power of a new alkoxylated polyol ester composition, it has been surprisingly observed that the produced sensory effect could be the result of a moisturizing action of these polymers, which was later confirmed by specific corneometry tests. It has been surprisingly noted that the idealized composition of alkoxylated polyol esters has the ability of maintaining the skin hydrated for up to 24 hours and is compatible with other ingredients commonly used in cosmetic products, which enables the use thereof in several cosmetic formulations, such as emulsions (W/O or O/W), micro-emulsions and nanoemulsions.

Thus, it is an object of the present invention to provide a new moisturizing composition comprising a mixture of esters of an alkoxylated polyol. Another object of the invention is to use this new composition in cosmetic formulations in order to increase skin hydration and consequently improve other properties, such as softness, shine and flexibility. Cosmetic formulations comprising this new moisturizing composition are also objects of the present invention. The advantages of the moisturizing composition of the present invention will be apparent from description that follows.

### BRIEF DESCRIPTION OF THE INVENTION

The present invention describes a new moisturizing composition comprising a mixture of esters of one or more alkoxylated polyols and finds use in the formulation of cosmetics for the skin. The composition of the present invention can be in the solid form or in the liquid form, in which case it also comprises one or more surfactants, emollients and, optionally, water. The moisturizing composition described herein is compatible with a large variety of emollients and additional ingredients used in cosmetic formulations, and has shown to be able to maintain the skin hydrated for 24 hours.

### BRIEF DESCRIPTION OF THE DRAWINGS

FIG. 1 shows the relative increase in skin hydration, shown as percentage (%) versus time (hours), obtained with the sample of Table 2.

### DETAILED DESCRIPTION OF THE INVENTION

The moisturizing composition referred to in the present invention comprises a mixture of esters of one or more alkoxylated polyols. Suitable polyols to be used in the present invention contain between 3 and 10 carbon atoms in total, and at least 3 free hydroxyls. From among the polyols that can be used, glycerol and derivatives thereof, such as di- and triglycerol, in addition to pentaerythritol, neopentylglycol, trimethylolpropane and sugar derivatives, such as mannitol, xylitol and sorbitol, or a mixture of these polyols can be mentioned. For the proper execution of the invention, the polyol must be alkoxylated until more than 120 moles of the alkoxide are obtained per mole of the polyol. The alkoxide can be ethylene oxide (EO), propylene oxide (PO), butylene oxide (BO) or a mixture of one or more of these oxides. The alkoxides react with hydroxyls of the polyols leading to the formation of long polymeric chains of the alkoxide used, which can be in the form of block or random copolymers when more than one alkoxide is used. The alkoxylated polyol is then esterified with one or more fatty acids that can have branched or linear, saturated or unsaturated chains and containing between 8 to 22 carbon atoms. Esterification generates a mixture that can contain di-, tri-, tetra-, penta- and hexaesters of the alkoxylated polyol. The methods for the preparation of compounds of this type are well established and were described in North American U.S. Pat. No. 4,983,329, U.S. Pat. No. 5,464,874 and U.S. Pat. No. 6,727,357, included herein by reference.

In a preferred embodiment of this invention, the polyol used is sorbitol, which is alkoxylated with EO until it has more than 120 moles of EO per mole of sorbitol. Next, the ethoxylated sorbitol is esterified with linear saturated fatty acids, either neat or in the form of mixtures, wherein caprylic acid, capric acid, lauric acid, myristic acid, palmitic acid and, preferably, stearic acid are the most suitable ones. Therefore, a composition comprising a mixture of ethoxylated sorbitol esters is obtained, wherein tetraesters and pentaesters are at a greater proportion than the total of esters based on the hydroxyl content and on the free acidity index in the composition, preferably forming more than 20 wt % of the total of esters. Such a composition is solid at room temperature, has high hydrating power and may be incorporated into various cosmetic formulations by heating, as practiced in conventional formulations.

Both the handling and the incorporation of the moisturizing composition described herein to various types of cosmetic formulations can be facilitated when the composition is in liquid state. In this case, for the composition to still maintain a high moisturizing power, the addition of one or more surfactants, solvents, and optionally water is recommended. Thus, in another preferred embodiment of this invention, the moisturizing composition is in liquid form and comprises from 35 to 60% by weight of a mixture of esters of one or more alkoxylated polyols containing more than 120 moles of alkoxide per each mole of polyol, in addition to 25 to 40% by weight of polyethylene glycol esters, 1 to 5% by weight of ethoxylated sorbitan esters, 0.5 to 2.0% by weight of glycols, and optionally water in an amount of up to 27% by weight. An example of a moisturizing composition in liquid form is shown in Table 1. Such a composition can be conveniently used in the preparation of various cosmetic formulations, including W/O and O/W emulsions, microemulsions, nanoemulsions or any other type of cosmetic emulsion in the form of a butter, cream, lotion or skin milk.

TABLE 1
Example moisturizing composition in liquid form.
Percentages by weight are based on the total of the composition.

| Components | Weight Percent |
|---|---|
| Mixture of ethoxylated sorbitol esters | 50% |
| Polyethylene glycol esters | 21% |
| Ethoxylated sorbitan esters | 2% |
| Propylene Glycol | 1% |
| Water (optional) | 26% |

The use of the composition of the present invention in liquid form to moisturize the skin and, consequently, increase its softness, shine and flexibility has several advantages. For example, due to the chemical nature of the chosen alkoxylated polyols, the moisturizing compositions in liquid form, such as that provided in Table 1, can be incorporated directly into cosmetic formulations without the need for heating or severe stirring, being easily homogenized. In an industrial scale, this operation is carried out in a tank provided with a stirrer, operating at room temperature. Thus, according to this invention, a moisturizing cosmetic formulation can be obtained by mixing one or more emulsifiers in a total of from 8 to 12% by weight of the formulation, one or more emollients also in a total of from 8 to 12% by weight of the formulation, from 1 to 5% by weight of the moisturizing composition of Table 1, and water q.s. An example of a cosmetic formulation comprising the moisturizing composition of the present invention is disclosed in Table 2.

TABLE 2
Example of a cosmetic formulation comprising
the moisturizing composition of Table 1 (PPG15 =
Polypropylene glycol (15) stearyl ether).

| | CONCENTRATION |
|---|---|
| COMPONENTS | (% by weight) |
| Etoxylated castor oil (emulsifier) | 3.0% |
| Span 80 (emulsifier) | 7.0% |
| Mineral oil (emollient) | 4% |
| PPG15 (emollient) | 6% |
| Moisturizing composition of Table 1 | 3.0% |
| Water | q.s.p. 100% |

Another advantage of the present invention is related to the chemical structure of the alkoxylated polyol esters employed. The long polymeric chains of the alkoxides, especially EO, form strong hydrogen bonds with water molecules, thus retaining moisture. However, the retained water molecules have lower mobility, which leads to a thickening of the formulation. Thus, the same mechanism that leads to hydration can also change viscosity of the formulation, in which case it will not require a thickener. In other words, in addition to imparting a moisturizing power to a cosmetic formulation, the incorporation of the composition of the present invention allows for controlling the viscosity of the formulation, which fact has not been observed in previous inventions (U.S. Pat. No. 4,687,843) and found to be a differential.

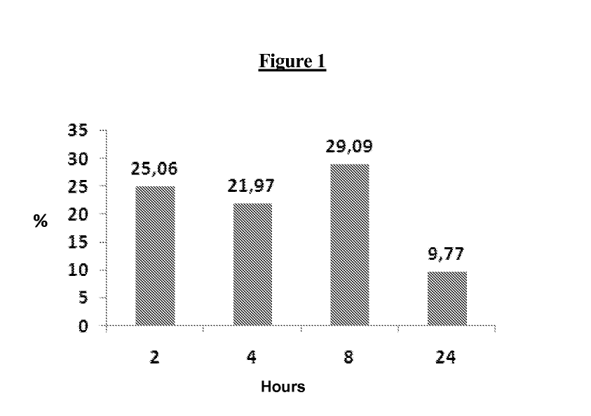

The moisturizing power of a cosmetic formulation can be easily verified by means of well-established techniques, such as corneometry (U. Heinrich, 2003, op. cit.), which determines the water content of the stratum corneum based on capacitance measurements of the skin. Corneometry assays were carried out with 20 volunteers using two formulations: one exactly as described in Table 2, and another similar one, but which lacked the moisturizing composition of Table 1. After accommodating volunteers for 60 minutes in an acclimated room, the study areas were defined as a rectangle of 10 cm² on each forearm of the volunteers. For each one of them, the formulation of Table 2 was applied to one of the forearms, while in the other, which served as a control, the formulation of Table 2 was applied without a moisturizing composition. A probe Cutometer MPA 580 coupled to the Multi Probe Adapter MPA 580 (CKeletronics, Germany) was used. Measurements of temperature of 22+/−2° C. and relative humidity of 50+/−5 UR were made 2, 4, 8 and 24 hours after the composition was applied on the forearm of each volunteer. FIG. 1 shows the average variation in the hydration percentage versus time on the forearm skin of 20 volunteers obtained based on the difference between measurements made on the area where the formulation containing the moisturizing composition was applied and those made on the control forearm, where a formulation with no moisturizing formulation was applied. In FIG. 1, the percentages represent, on average, how much more hydrated was the area that received the formulation containing the moisturizing composition compared to the control. It is clearly observed that hydration of the area on which the formulation described in Table 2 was applied exceeded that of the control by more than 20% within 8 hours after application, when it reached a maximum of 29.09%. Even after 24 hours, the skin of the forearm on which the formulation containing the moisturizing composition was applied was still about 10% more hydrated than the skin of the control forearm.

Further observations have shown that, in addition to not irritating the volunteers' skin, the moisturizing compositions obtained in accordance with the present invention have pleasant sensory properties, meeting the market's demands.

1. A moisturizing composition comprising a mixture of di-, tri-, tetra-, penta- and hexa esters of an alkoxylated polyol having more than 120 moles of alkoxide per mole of polyol.

2. The moisturizing composition of claim 1, wherein the alkoxylated polyol esters are obtained from saturated or unsaturated fatty acids comprising between 8 to 22 carbon atoms.

3. The moisturizing composition of claim 1, wherein the alkoxylated polyol is obtained by combining an alkoxide selected from the group consisting of ethylene oxide, propylene oxide, butylene oxide, and mixtures thereof, with a polyol selected from the group consisting of pentaerythritol, neopentylglycol, trimethylolpropane, glycerol, one or more glycerol derivatives, one or more sugar derivatives, and mixtures thereof.

4. The moisturizing composition of claim 1, wherein the alkoxylated polyol is ethoxylated sorbitol having more than 120 moles of ethylene oxide per mole of sorbitol.

5. The moisturizing composition of claim 1, further comprising one or more solvents, surfactants and, optionally, water.

6. The moisturizing composition of claim 5, wherein the solvents are glycols, and the surfactants are selected from the group consisting of polyethylene glycol esters, ethoxylated sorbitan esters, and mixtures thereof.

7. The moisturizing composition of claim 5, wherein the moisturizing composition comprises 35 to 60 wt % of a mixture of esters of an alkoxylated polyol containing more than 120 moles of the alkoxide per mole of the polyol, 25 to 40 wt % of polyethylene glycol esters, 1 to 5 wt % of ethoxylated sorbitan esters, 0.5 to 2.0% by weight of glycols and, optionally, up to 27% by weight of water.

8. A method of using a moisturizing composition for moisturizing skins, the method comprising preparing a cosmetic formulation containing the moisturizing composition of claim 1.

9. The method of claim 8, wherein the alkoxylated polyol is ethoxylated sorbitol.

10. A cosmetic formulation comprising:
(i) 8 to 12% by weight of one or more emulsifiers;
(ii) 8 to 12% by weight of one or more emollients;
(iii) 1 and 5 wt % of a moisturizing composition, wherein the moisturizing composition comprises 35 to 60wt % of a mixture of alkoxylated polyol esters containing more than 120 moles of the alkoxide per mole of the polyol, 25 to 40 wt % of polyethylene glycol esters, 1 to 5 wt % of ethoxylated sorbitan esters, 0.5 to 2.0% by weight of glycols, and optionally up to 27% by weight of water; and
(iv) water q.s.

11. The formulation of claim 10, wherein the alkoxylated polyol esters comprise ethoxylated sorbitol esters obtained from stearic acid.

12. The formulation of claim 10, wherein the emulsifier comprises one or more ethoxylated vegetable oils, one or more sorbitan derivatives, or mixtures thereof.

13. The formulation of claim 10, wherein the emollient comprises one or more mineral oils, one or more glycol derivatives, or mixtures thereof.

14. The formulation of claim 10, wherein the formulation is a W/O emulsion, a O/W emulsion, a microemulsion or a nanoemulsion.

15. The moisturizing composition of claim 1, wherein the weight percentage of the tetra- and pentaesters of the alkoxylated polyol is at least 20% by weight of the total alkoxylated polyol esters.

16. The moisturizing composition of claim 2, where the fatty acid is stearic acid.

17. The method of claim 9, wherein the esters are obtained from stearic acid.
